(19) United States
(12) Patent Application Publication
(10) Pub. No.: US 2023/0165519 A1
(43) Pub. Date: Jun. 1, 2023

(54) SLEEP SCREENING DATA MANAGEMENT SYSTEM AND SLEEP SCREENER MANAGEMENT SYSTEM

(71) Applicant: BOE Technology Group Co., Ltd., Beijing (CN)

(72) Inventors: Guilong YANG, Beijing (CN); Zigang LIU, Beijing (CN)

(21) Appl. No.: 17/921,894

(22) PCT Filed: May 20, 2021
(86) PCT No.: PCT/CN2021/094864

§ 371 Date: Oct. 27, 2022

(30) Foreign Application Priority Data
May 20, 2020 (CN) 202010428203.6

(51) Int. Cl. A61B 5/00; G16H 40/63; G16H 15/00

(52) U.S. Cl. CPC A61B 5/4806; G16H 40/63; G16H 15/00; H04W 76/10

(57) ABSTRACT

A sleep screening data management system includes a sleep screener management device (01). The sleep screener management device (01) is configured to obtain sleep monitoring data collected by sleep screeners (011) managed thereby, and to send the sleep monitoring data wirelessly to a data obtaining device (02). The data obtaining device (02) is configured to send the obtained sleep monitoring data to a data analysis device (03). The data analysis device (03) analyzes and processes the received sleep monitoring data, and generates a sleep screening report. The sleep screening data management system uses the sleep screener management device (01) and the data obtaining device (02) to send sleep monitoring data to the data analysis device (03), thereby acquiring a sleep screening report without requiring operations by a doctor, and realizing remote diagnosis functions.

### CROSS REFERENCE TO RELATED APPLICATIONS

The present disclosure is a national phase entry under 35 U.S.C. § 371 of International Application No. PCT/CN2021/094864, filed on May 20, 2021, which claims the priority of Chinese Patent Application No. 202010428203.6, filed with the China National Intellectual Property Administration on May 20, 2020 and entitled “SLEEP SCREENING DATA MANAGEMENT SYSTEM AND SLEEP SCREENER MANAGEMENT SYSTEM”, the entire content of which are incorporated herein by reference.

### FIELD

The present disclosure relates to the technical field of medical treatment and health, in particular to a sleep screening data management system and a sleep screener management system.

### BACKGROUND

Sleep-disordered breathing refers to hypopnea or apnea caused by increased upper airway resistance or respiratory center drive disorder, which leads to a series of pathophysiological changes and clinical symptoms, which is divided into obstructive sleep apnea hypopnea syndrome and central sleep apnea syndrome.

Generally, during the diagnosis of sleep-disordered breathing, blood oxygen and heart rate change data of a patient during sleep are collected by a sleep screener, then a doctor sends the data to a respiratory rehabilitation system to get a sleep screening report, and then the doctor determines whether the patient has a sleep breathing disease or not according to the sleep screening report. However, at present, sleep screening reports are available only through doctors.

### SUMMARY

A sleep screening data management system provided by embodiments of the present disclosure includes: at least one sleep screener management device, a data obtaining device and a data analysis device.

The sleep screener management device is configured to obtain sleep monitoring data collected by sleep screeners managed by the sleep screener management device, and send the sleep monitoring data wirelessly to the data obtaining device.

The data obtaining device is configured to send the obtained sleep monitoring data to the data analysis device.

The data analysis device analyzes and processes the received sleep monitoring data to generate a sleep screening report.

Optionally, in the sleep screening data management system provided by the embodiments of the present disclosure, the sleep screener management device includes: a plurality of sleep screeners, a Bluetooth gateway and a wireless router.

The Bluetooth gateway is configured to establish a connection with the plurality of sleep screeners, obtain sleep monitoring data collected by each sleep screener, and send the obtained sleep monitoring data to the data obtaining device in a preset format through the wireless router.

Optionally, in the sleep screening data management system provided by the embodiments of the present disclosure, the sleep screener management device further includes a charger configured to charge the sleep screeners.

Optionally, in the sleep screening data management system provided by the embodiments of the present disclosure, the data obtaining device includes: a Bluetooth gateway access controller and a Bluetooth device data collection management device.

The Bluetooth gateway access controller is configured to establish a connection with each sleep screener management device.

The Bluetooth device data collection management device is configured to remotely establish data communication with each sleep screener management device, so as to obtain the sleep monitoring data sent by the sleep screener management device in the preset format, and send the received sleep monitoring data in the preset format to the data analysis device.

Optionally, in the sleep screening data management system provided by the embodiments of the present disclosure, the data analysis device is configured to:

process the received sleep monitoring data in the preset format to generate a formatted file; and
analyze and process the generated formatted file according to a preset algorithm to generate the sleep screening report.

Optionally, in the sleep screening data management system provided by the embodiments of the present disclosure, the data analysis device is further configured to send the generated sleep screening report to terminal equipment.

Optionally, in the sleep screening data management system provided by the embodiments of the present disclosure, the wireless router is a 4G router or a 5G router.

Correspondingly, embodiments of the present disclosure further provide a sleep screener management system, including: a plurality of sleep screeners, a Bluetooth gateway and a wireless router.

The sleep screeners are configured to collect sleep monitoring data.

The Bluetooth gateway is configured to establish a connection with the plurality of sleep screeners, and obtain the sleep monitoring data collected by each sleep screener.

The wireless router is configured to transmit the sleep monitoring data obtained by the Bluetooth gateway.

Optionally, the sleep screener management system provided by the embodiments of the present disclosure further includes a charger configured to charge the sleep screeners.

Optionally, in the sleep screener management system provided by the embodiments of the present disclosure, the wireless router is a 4G router or a 5G router.

### BRIEF DESCRIPTION OF THE DRAWINGS

FIG. 1 is a schematic structural diagram of a sleep screening data management system provided by an embodiment of the present disclosure.

FIG. 2 is another schematic structural diagram of a sleep screening data management system provided by an embodiment of the present disclosure.

FIG. 3 is another schematic structural diagram of a sleep screening data management system provided by an embodiment of the present disclosure.

FIG. 4 is a schematic structural diagram of a sleep screener management system provided by an embodiment of the present disclosure.

### DETAILED DESCRIPTION OF THE EMBODIMENTS

In order to enable the above objectives, features and advantages of the present disclosure more clearly and easy to understand, the present disclosure will be further illustrated in combination with accompanying drawings and embodiments. However, exemplary implementations can be implemented in many forms, and should not be understood to be limited to the implementation described herein. On the contrary, these implementations are provided to enable the present disclosure more thorough and complete, and to fully convey the concept of example implementations to those skilled in the art. The same accompanying drawing reference numerals in the figures indicate the same or similar structure, and thus repeated descriptions thereof will be omitted. The words expressing position and direction described in the present disclosure are all illustrated with the accompanying drawings as an example, but can be changed as needed, and the changes are included in the protection scope of the present disclosure. The accompanying drawings of the present disclosure are only configured to indicate a relative position relationship and do not represent a true scale.

It should be noted that details are set forth in the following descriptions to facilitate a full understanding of the present disclosure. However, the present disclosure can be implemented in many ways different from those described herein, and those skilled in the art can make similar popularization without violating the connotation of the present disclosure.

Therefore, the present disclosure is not limited by the embodiments disclosed below. The subsequent description of the specification shows preferred embodiments for implementing the present discourse, but the purpose of the description is to describe the general principles of the present disclosure and not to limit the scope of the present disclosure. The scope of protection of the present disclosure shall be as defined in the attached claims.

The sleep screening data management system and the sleep screener management system provided by the embodiments of the present disclosure are described in detail below in combination with the attached drawings.

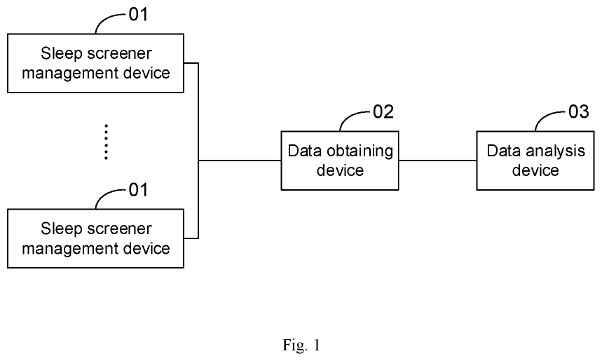

The sleep screening data management system provided by the embodiments of the present disclosure, as shown in FIG. 1, includes: at least one sleep screener management device 01, a data obtaining device 02 and a data analysis device 03.

The sleep screener management device 01 is configured to obtain sleep monitoring data collected by sleep screeners managed thereby and send the sleep monitoring data wirelessly to the data obtaining device 02.

The data obtaining device 02 is configured to send the obtained sleep monitoring data to the data analysis device 03.

The data analysis device 03 analyzes and processes the received sleep monitoring data to generate a sleep screening report.

According to the sleep screening data management system provided by the embodiments of the present disclosure, the sleep screener management device is configured to obtain the sleep monitoring data collected by the sleep screeners managed thereby, and send the sleep monitoring data wirelessly to the data obtaining device; the data obtaining device is configured to send the obtained sleep monitoring data to the data analysis device; and the data analysis device analyzes and processes the received sleep monitoring data to generate the sleep screening report. The sleep screening data management system provided by the embodiments of the present disclosure uses the sleep screener management devices and the data obtaining device to send the sleep monitoring data to the data analysis device, thereby obtaining the sleep screening report without requiring operations by a doctor, and realizing remote diagnosis functions.

During implementations, the sleep screener management devices may be distributed in hospitals. Each sleep screener management device is configured to centrally manage sleep screeners in a hospital where the each sleep screener management device is located.

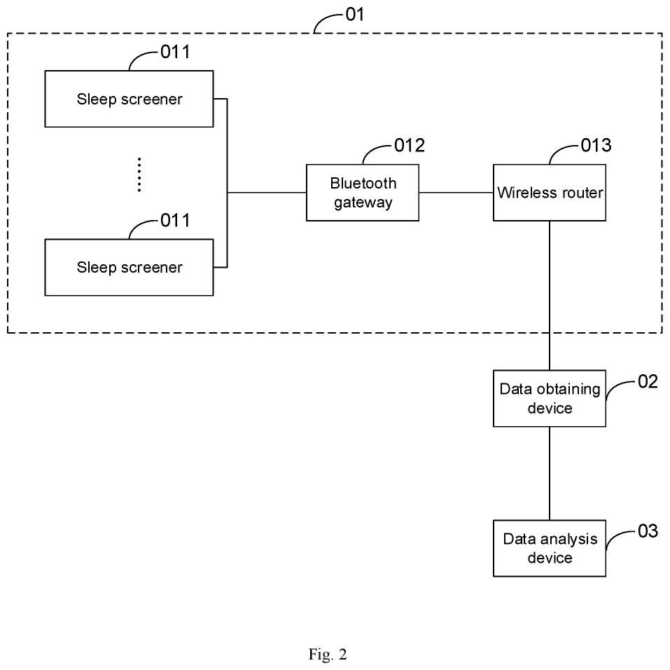

Optionally, in the sleep screening data management system provided by the embodiments of the present disclosure, as shown in FIG. 2, the sleep screener management device 01 includes: a plurality of sleep screeners 011, a Bluetooth gateway 012 and a wireless router 013.

The Bluetooth gateway 012 is configured to establish a connection with the plurality of sleep screeners 011, obtain sleep monitoring data collected by each sleep screener 011, and send the obtained sleep monitoring data to the data obtaining device 02 in a preset format through the wireless router 013.

During implementations, in the sleep screening data management system provided by the embodiments of the present disclosure, the wireless router can be a 4G router or a 5G router to realize remote wireless communication of data.

Optionally, in the sleep screening data management system provided by the embodiments of the present disclosure, the sleep screener management device 01 further includes a charger configured to charge the sleep screeners 011. In this way, the sleep screeners 011 can be charged when being placed in the sleep screener management device 01.

During implementations, in the sleep screening data management system provided by the embodiments of the present disclosure, the quantity of the chargers in the sleep screener management device 01 is not limited. For example, the quantity of the chargers may be the same as the quantity of the sleep screeners 011, such that all the sleep screeners 011 may be ensured to be charged concurrently. Further, the quantity of the chargers may also be less than the quantity of the sleep screeners 011.

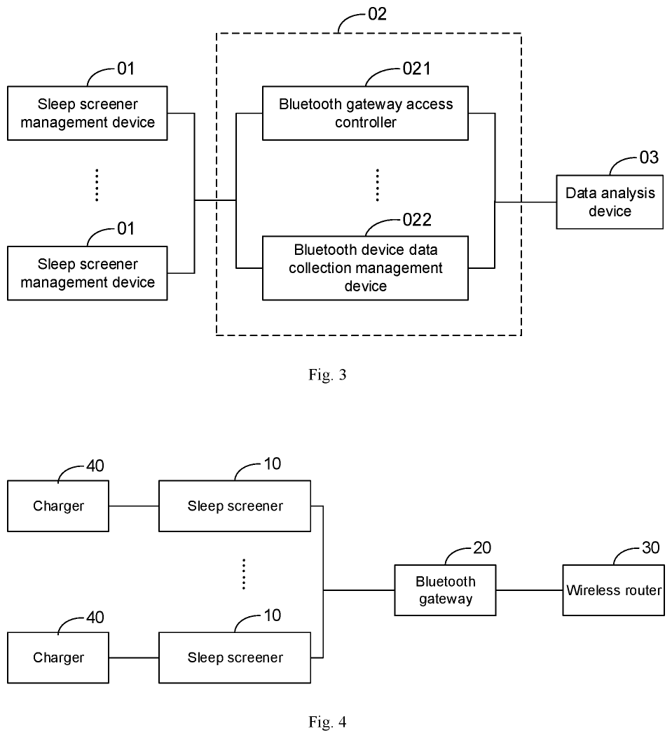

Optionally, in the sleep screening data management system provided by the embodiments of the present disclosure, as shown in FIG. 3, the data obtaining device 02 may include: a Bluetooth gateway access controller 021 and a Bluetooth device data collection management device 022.

The Bluetooth gateway access controller 021 is configured to establish a connection with each sleep screener management device 01.

The Bluetooth device data collection management device 022 is configured to remotely establish data communication with each sleep screener management device 01, obtain sleep monitoring data sent by the sleep screener management devices 01 in a preset format, and send the sleep monitoring data in the preset format to the data analysis device 03.

Optionally, in the sleep screening data management system provided by the embodiments of the present disclosure, the data analysis device 03 is configured to:

process the sleep monitoring data in the preset format to generate a formatted file; and
analyze and process the generated formatted file according to a preset algorithm to generate the sleep screening report.

Optionally, in the sleep screening data management system provided by the embodiments of the present disclosure, the data analysis device is further configured to send the generated sleep screening report to terminal equipment.

During implementations, the sleep screening data management system provided by the embodiments of the present disclosure may be configured to be applied into medical diagnosis of hospitals. The sleep screening data management system provided by the embodiments of the present disclosure is illustrated below in combination with application scenarios. The working processes are as follows.

(1) in the process of detecting a patient, a doctor orders a sleep screening list if he or she thinks the patient needs sleep screening.

(2) a user takes the sleep screening list to a sleep screener management device of a nurse station to get a sleep screener.

(3) the user needs to scan a two-dimensional code of the sleep screener, register user information and bind a relationship between the sleep screener and a user account to ensure subsequent sleep monitoring data collected by the sleep screener and a subsequent generated sleep screening report can be attributed to the name of the user.

(4) the user takes the sleep screener away and wears the sleep screener for normal sleep to complete the collection of sleep monitoring data.

(5) the user returns the sleep screener to the location of the sleep screener management device in the hospital and plugs in a charger.

(6) a Bluetooth gateway of the sleep screener management device automatically finds the sleep screener and establishes a connection with the sleep screener to obtain the corresponding sleep monitoring data.

(7) a wireless router of the sleep screener management device sends the sleep monitoring data to a data analysis device through a data obtaining device.

(8) the data analysis device generates a sleep screening report according to the obtained sleep monitoring data of the user and pushes the sleep screening report to a doctor management end and a user WeChat end.

(9) the doctor reviews the sleep screening report of the designed user and further determines the patient's disease status based on the report.

(10) the user views his/her sleep screening report through a WeChat Official Account.

Based on the same inventive concept, embodiments of the present disclosure further provide a sleep screener management system, as shown in FIG. 4, including: a plurality of sleep screeners 10, a Bluetooth gateway 20 and a wireless router 30.

The sleep screeners 10 are configured to collect sleep monitoring data.

The Bluetooth gateway 20 is configured to establish a connection with the plurality of sleep screeners 10, and obtain the sleep monitoring data collected by each sleep screener 10.

The wireless router 30 is configured to transmit the sleep monitoring data obtained by the Bluetooth gateway 20.

The sleep screener management system provided by the embodiments of the present disclosure uses the Bluetooth gateway to automatically scan and find the sleep screener(s) and establish a connection with the sleep screener(s) to obtain the sleep monitoring data collected by the sleep screener(s). Wireless communication of the sleep monitoring data is realized through the wireless router. Manual operation can be eliminated to realize remote diagnosis.

Optionally, the sleep screener management system provided by the embodiments of the present disclosure, as shown in FIG. 4, may further include: a charger(s) 40 configured to charge the sleep screeners 10.

Optionally, in the sleep screener management system provided by the embodiments of the present disclosure, the wireless router 30 may be a 4G router or a 5G router, which is not limited here.

According to the sleep screening data management system and the sleep screener management system provided by the embodiments of the present disclosure, the sleep screener management device is configured to obtain the sleep monitoring data collected by the sleep screeners managed thereby, and send the sleep monitoring data wirelessly to the data obtaining device; the data obtaining device is configured to send the obtained sleep monitoring data to the data analysis device; and the data analysis device analyzes and processes the received sleep monitoring data to generate the sleep screening report. The sleep screening data management system provided by the embodiment of the present disclosure uses the sleep screener management device and the data obtaining device to send the sleep monitoring data to the data analysis device, thereby obtaining the sleep screening report without requiring operations by a doctor, and realizing remote diagnosis functions.

Apparently, those skilled in the art can make various changes and transformations to the present disclosure without departing from the spirit and scope of the present disclosure. In this case, if these changes and transformations of the present disclosure belong to the scope of claims and their equivalents of the present disclosure, the present disclosure also intends to include these changes and transformations.

What is claimed is:

1. A sleep screening data management system, comprising:
at least one sleep screener management device,
a data obtaining device, and
a data analysis device;
wherein the sleep screener management device is configured to:
obtain sleep monitoring data collected by sleep screeners managed by the sleep screener management device, and
send the sleep monitoring data wirelessly to the data obtaining device;
the data obtaining device is configured to send the sleep monitoring data to the data analysis device; and
the data analysis device is configured to analyze and process the sleep monitoring data to generate a sleep screening report.

2. The sleep screening data management system of claim 1, wherein the sleep screener management device comprises:
the plurality of sleep screeners,
a Bluetooth gateway, and
a wireless router;
wherein the Bluetooth gateway is configured to:
establish a connection with the plurality of sleep screeners,
obtain the sleep monitoring data collected by the sleep screeners, and
send the sleep monitoring data to the data obtaining device in a preset format through the wireless router.

3. The sleep screening data management system of claim 2, wherein the sleep screener management device further comprises:
a charger configured to charge the sleep screeners.

4. The sleep screening data management system of claim 2, wherein the data obtaining device comprises:
a Bluetooth gateway access controller, and
a Bluetooth device data collection management device;
wherein the Bluetooth gateway access controller is configured to establish a connection with the sleep screener management device; and
the Bluetooth device data collection management device is configured to:
remotely establish data communication with the sleep screener management device,
obtain the sleep monitoring data sent by the sleep screener management device in the preset format, and
send the sleep monitoring data in the preset format to the data analysis device.

5. The sleep screening data management system of claim 4, wherein the data analysis device is configured to:
process the sleep monitoring data in the preset format to generate a formatted file; and
analyze and process the formatted file according to a preset algorithm to generate the sleep screening report.

6. The sleep screening data management system of claim 4, wherein the data analysis device is further configured to send the sleep screening report to terminal equipment.

7. The sleep screening data management system of claim 2, wherein the wireless router is:
a 4G router, or
a 5G router.

8. A sleep screener management system, comprising:
a plurality of sleep screeners,
a Bluetooth gateway, and
a wireless router;
wherein the sleep screeners are configured to collect sleep monitoring data;
the Bluetooth gateway is configured to:
establish a connection with the plurality of sleep screeners, and
obtain the sleep monitoring data collected by the sleep screeners; and
the wireless router is configured to transmit the sleep monitoring data obtained by the Bluetooth gateway.

9. The sleep screener management system of claim 8, further comprising:
a charger configured to charge the sleep screeners.

10. The sleep screener management system of claim 9, wherein the wireless router is:
a 4G router, or
a 5G router.
